(19) United States
(12) Patent Application Publication
(10) Pub. No.: US 2025/0375215 A1
(43) Pub. Date: Dec. 11, 2025

(54) SYSTEMS AND METHODS FOR LONG HAIR TRANSPLANTATION

(71) Applicant: The General Hospital Corporation, Boston, MA (US)

(72) Inventors: William Farinelli, Boston, MA (US); Sandeep Korupolu, Boston, MA (US); Walfre Franco, Boston, MA (US); Lynn Drake, Boston, MA (US); Richard Rox Anderson, Boston, MA (US)

(21) Appl. No.: 19/127,809

(22) PCT Filed: Nov. 8, 2023
(86) PCT No.: PCT/US2023/037023

§ 371 Date: May 7, 2025

Related U.S. Application Data
(60) Provisional application No. 63/382,905, filed on Nov. 9, 2022.

(51) Int. Cl. A61B 17/3205; A61B 17/00; A61B 17/34

(52) U.S. Cl. CPC A61B 17/32053; A61B 17/3468; A61B 2017/00526; A61B 2017/00752; A61B 2017/3454

(57) ABSTRACT

Hair transplant systems and methods include or use a casing; an extracting needle having a first groove extending longitudinally from a distal end of the extracting needle, the first groove including a notch; a lock disposed on the extracting needle and configured to selectively close at least a portion of the first groove; and a pin extending through a center of the casing and configured to cause the extracting needle to selectively take in or eject a hair follicle.

### CROSS-REFERENCE TO RELATED APPLICATIONS

This application claims the benefit of and priority to U.S. Provisional Patent Application No. 63/382,905. filed on Nov. 9, 2022, entitled “Long Hair Transplantation,” the entire contents of which are herein incorporated by reference for all purposes.

### TECHNICAL FIELD

This disclosure relates to the field of hair transplantation. More specifically, this disclosure relates to systems and methods for the transplantation of long hair.

### BACKGROUND

Hair loss is one of the most psychological issues that is affecting 80 million people in the United States alone. As a result, the commercial market for addressing hair loss is a multi-billion dollar industry, from drug therapies to hair transplantation.

Hair transplantation is a procedure that involves implanting multiple hair follicles or follicular units, from a donor site of a donor, into a recipient site of a patient. Prior to the procedure, in comparative examples the hair at the donor site must be shaved to a length of approximately 2 millimeters (mm) or shorter. Generally, the procedure is then done by first identifying a hair follicle in the donor site, coring around the hair follicle, and removing the hair follicle from the donor site. Then, a small opening is created in the recipient site. After the small opening has been created the hair follicle is implanted within the opening, and the opening is allowed to heal around the implanted hair follicle.

Comparative examples of hair transplant surgery typically succeed in transferring nearly 100% of viable hair follicles, which then grow hair according to the biological fate of the donor site, not the recipient site. On the scalp, generally over 90% of hair follicles are in the actively growing anagen phase. Upon transplantation, most follicles lose the actively growing hair and then transition into a month-long catagen phase in which active growth abates and the lower third of the follicle undergoes loss by apoptosis. After several more weeks, a new anagen phase begins to produce new hair, and the transplanted hair is shed as the new hair emerges. Alternatively, the freshly transplanted hair stops growing due to anagen arrest and enters a dystrophic anagen phase that produces a thin and fragile hair shaft before recovery. Either way, the transplanted hair is lost.

### SUMMARY

The present disclosure overcomes these and other drawbacks by providing systems and methods for hair transplantation of long hair using a single modular device which includes both extraction and implantation features. In some examples, the systems and methods described herein eliminate intermediate stages in the hair transplantation process (e.g., manipulating hair follicles such as by placing them in the holding location prior to implant). Accordingly, the present disclosure provides for systems and methods of hair transplantation which may proceed much more quickly and efficiently than comparative methods. The systems and methods of the present disclosure are provided for improved hair transplant procedures that increase extraction speed, opening speed, and implantation speed, thereby increasing efficacy and reducing cost. Additionally, unlike comparative procedures, the systems and methods of the present disclosure are capable of transplanting long (e.g., ≥1 mm) hair. Moreover, by reducing the time between extraction and implantation, the systems and methods described herein reduce stress on the hair follicles, which may lead to better transplantation outcomes.

According to one aspect of the present disclosure, a hair transplant system is provided. The hair transplant system comprises a casing; an extracting needle having a first groove extending longitudinally from a distal end of the extracting needle, the first groove including a notch; a lock disposed on the extracting needle and configured to selectively close at least a portion of the first groove; and a pin extending through a center of the casing and configured to cause the extracting needle to selectively take in or eject a hair follicle.

According to another aspect of the present disclosure, a hair transplant method is provided. The method comprises providing a hair transplant system comprising an extracting needle having a first groove extending longitudinally from a distal end of the extracting needle, the first groove including a notch; inserting a hair through the first groove and out the notch; engaging a lock disposed on the extracting needle, thereby to close at least a portion of the first groove; extracting a follicle of the hair by piercing a donor site with the extracting needle; attaching an implantation to the hair transplant system, the implantation unit including an implanting needle and a second groove configured to align with the first groove in a state of the implantation unit being attached to the hair transplant system; implanting the follicle by piercing a recipient site with the implanting needle; and disengaging the lock.

According to another aspect of the present disclosure a method of manufacturing a hair transplant system is provided. The method comprises providing an extracting unit, the extracting unit including: a casing, an extracting needle having a first groove extending longitudinally from a distal end of the extracting needle, the first groove including a notch, lock disposed on the extracting needle and configured to selectively close at least a portion of the first groove, and a pin extending through a center of the casing and configured to cause the extracting needle to selectively take in or eject a hair follicle; and providing an implantation unit, the implantation unit including an implanting needle and a second groove, wherein the implantation unit is configured to be removably coupled to the casing such that, in a state of the implantation unit being coupled to the casing, the second groove is aligned with the first groove.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an example in more detail than is necessary for a fundamental understanding of the teachings of the disclosures. In the drawings:

FIG. 1A is a perspective view of an example of a hair transplant system including an implantation unit and extraction unit in accordance with one aspect of the present disclosure.

FIG. 1B is an exploded view of the hair transplant system of FIG. 1A.

FIG. 2 is a perspective view of a portion of the hair transplant system of FIG. 1A.

FIG. 3A is a side view of an example of an extracting needle and lock in accordance with one aspect of the present disclosure.

FIG. 3B is a side view of an example of an extracting needle and lock in accordance with one aspect of the present disclosure.

FIG. 4 is a side view of an example of an implantation unit in accordance with one aspect of the present disclosure

FIG. 5 is a flowchart describing an example method of operation of the hair transplantation system.

FIG. 6 is a flowchart describing an example method of extraction.

FIG. 7 is a schematic of an example of an automated hair transplant system in accordance with various aspects of the present disclosure.

FIG. 8 is a flowchart describing an example method of manufacturing and assembly, according to various aspects of the present disclosure.

### DETAILED DESCRIPTION

In the following detailed description, reference is made to the accompanying drawings in which specific examples are shown by way of illustration. These examples are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

Unless otherwise indicated, the various features illustrated in the drawings may not be drawn to scale. The illustrations presented herein are not necessarily intended to be actual views of any particular method, device, or system, but are merely idealized representations that are employed to describe various embodiments of the disclosure. Accordingly, the dimensions of the various features as illustrated may be arbitrarily expanded or reduced for clarity. In addition, some of the drawings may be simplified for clarity. Thus, the drawings may not depict all of the components of a given apparatus (e.g., device) or method.

In addition, like reference numerals may be used to denote like features throughout the specification and figures.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “only one of,” or “exactly one of.” For example, a list of “only one of A, B, or C” indicates options of: A, but not B and C; B, but not A and C; and C, but not A and B. In contrast, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more A, one or more B, and one or more C. Similarly, a list preceded by “a plurality of” (and variations thereon) and including “or” to separate listed elements indicates options of one or more of each of multiple of the listed elements. For example, the phrases “a plurality of A, B, or C” and “two or more of A, B, or C” indicate options of: one or more A and one or more B; one or more B and one or more C; one or more A and one or more C; and one or more A, one or more B, and one or more C.

As will be described herein, the present disclosure provides systems and methods for transplanting hair follicles from a donor site to a recipient site using a long hair transplant system and method. The long hair transplant system described herein include an extracting unit configured to extract at least one hair follicle from a donor site and an implantation unit removably coupled to the extracting unit, the implantation unit including configured to form an opening into a recipient site.

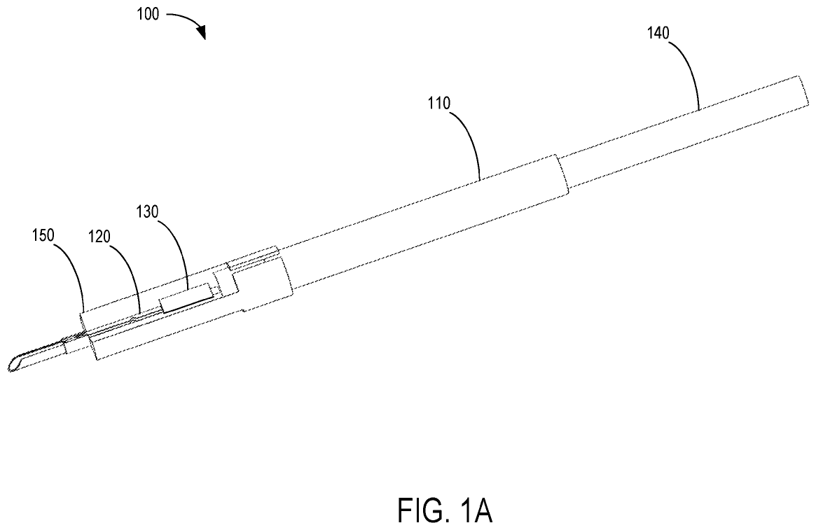

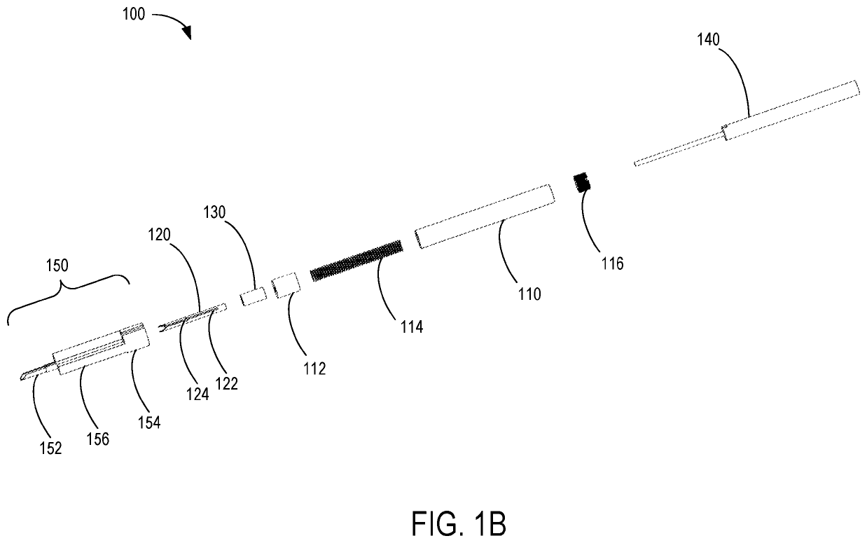

Referring now to the drawings wherein like reference numerals correspond to similar elements throughout the several views and, more specifically, referring to FIGS. 1A and 1B, a hair transplant system 100 for extracting hair follicles from a donor site of a donor and implanting them into a recipient site of a patient is illustrated. These hair follicles, or follicular units, can contain a single hair or multiple hairs grouped together. The donor and patient may be the same or different persons. In some examples, the donor site is a portion of the scalp located on the rear of a patient's head, and the recipient site is a portion of the scalp located on the top and/or sides of the same patient's head.

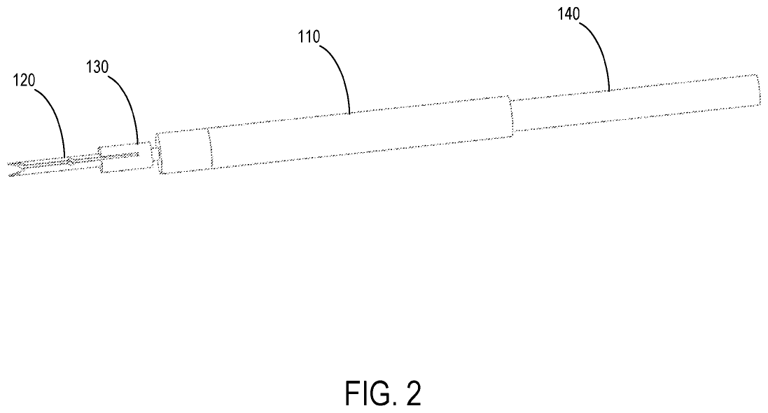

As can be seen in the illustration of FIG. 1A, the hair transplant system 100 includes an extracting unit including a casing 110, an extracting needle 120, a lock 130, and a pin 140; and an implantation unit 150 removably coupled to the extracting unit. The implantation unit 150 is configured to form an opening into a recipient site. With the implantation unit 150 removed from the extracting unit (see FIG. 2), the hair transplant system 100 is configured to perform extraction. With the implantation unit 150 coupled to the extracting unit (see FIG. 1A), the hair transplant system 100 is configured to perform implantation. FIG. 1A illustrates the hair transplant system 100 in an assembled view; and FIG. 1B illustrates the hair transplant system 100 in an exploded view.

As can be seen in the exploded view of FIG. 1B, the casing 110 includes a mounting collar 112 attached to a distal end thereof and configured to receive the extracting needle 120, a resilient element 114 received in the casing 110, and a pin stop 116 attached to a proximal end thereof. As used herein, the terms “proximal” and “distal” refer to the position along a longitudinal axis of the hair transplant system 100, from the perspective of an operator of the hair transplant system. Thus, in use, the proximal end portion is located nearer to the operator, and the distal end portion is located nearer to the patient.

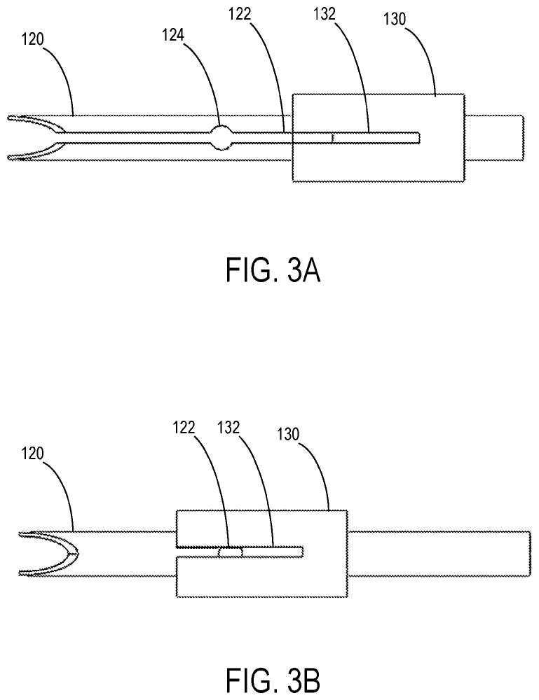

Referring now to FIGS. 1B and 3A-B, the extracting needle 120 includes a first groove 122 that extends longitudinally from a distal end of the extracting needle 120 to a point relatively near (but not necessarily at) a proximal end of the extracting needle 120. The first groove 122 includes a notch 124. A lock 130 is disposed on the extracting needle 120. The lock 130 may be a cylindrical collar configured to receive the extracting needle 120 therein. The lock 130 is configured to slide along the extracting needle 120 between a first (unlocked) position, as shown in FIG. 3A, in which the lock 130 is longitudinally displaced from the notch 124 (e.g., in the proximal direction) such that the notch 124 is uncovered; and a second (locked) position, as shown in FIG. 3B, in which the lock 130 has a groove 132 that facilitates the movement of hair therethrough. The notch 124 may be circular in shape. In cooperation with the first groove 122, the notch 124 may be configured to receive a long (e.g., ≥1 mm, and more particularly on the order of cm or tens of cm) hair during an extraction process and to ensure that the hair is under tension during extraction. While FIGS. 3A-3B illustrate an example in which the lock 130 has a groove 132, in other examples the lock may have no groove and may travel up to the notch 122 without covering the notch 122, thereby to achieve the second (locked) position.

The extracting needle 120 may be a coring needle, formed as a hollow needle with a coring needle lumen extending therethrough. Further, the coring needle may include a first distal cutting end. The distal cutting end is configured to cut into the donor site to form a core in order to extract a hair follicle within the core. Each distal cutting end may include a pair of angled surfaces that angle toward each other, intersecting at the distal end of the coring needle. Accordingly, the pair of angled surfaces form a pair of cutting edges disposed on opposite sides of the coring needle lumen. The pair of cutting edges are effectively aligned across the coring needle, such that they both extend radially from an inner surface of the coring needle to an outer surface of the coring needle. As such, the coring needle can be configured to cut into tissue by driving the coring needle into the tissue, without needing to rotate the coring needle.

Returning to FIG. 1B, the pin 140 extends through a center of the casing 110. The pin 140 is configured to cause the extracting needle 120 to selectively take in or eject a hair follicle. The pin 140 includes a distal end and a proximal end. The distal end of the pin 140 is covered with a mesh (not shown), and the proximal end of the pin 140 may have a port to allow a connection to a vacuum source. The mesh can be configured to prevent a core extracted from the donor site from being pulled into a central lumen of the pin 140. For example, if suction is applied to the central lumen (e.g., via the port using the vacuum source), the mesh can provide a surface that allows suction therethrough, but does not allow the passage of objects such as the extracted core. The mesh can define a porous surface including a plurality of holes. The distal end of the pin 140 is inserted into the casing 110 of the extracting unit. The resilient element 114 is configured to provide a return force such that, if the pin 140 is pressed into the casing 110, the resilient element 114 causes the pin 140 to return to its original position. The pin stop 116 of the casing 110 may be configured to prevent the pin 140 from falling out of the casing 110. As illustrated in FIG. 1B, the resilient element 114 is a spring. However, in other implementations the resilient element 114 may be another device that exerts a biasing force.

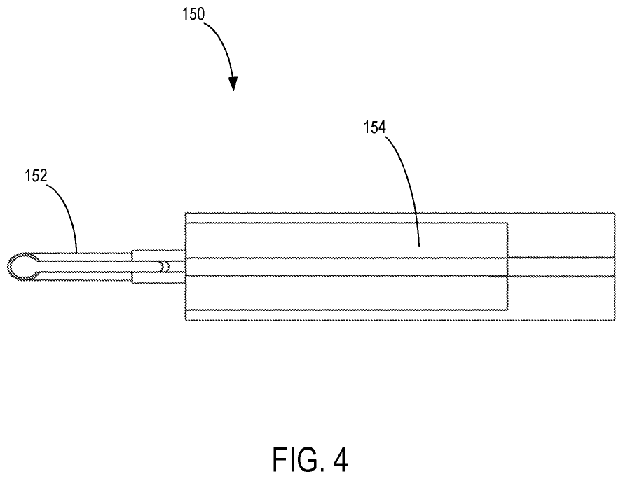

Referring now to FIGS. 1B and 4, the distal end of the implantation unit 150 includes an implanting needle 152. The implanting needle 152 may include an interior flange configured to rest on a tip of the extracting needle 120 when the implantation unit 150 is attached to the extraction needle 120. The central longitudinal portion of the implantation unit 150 includes a second groove 154. The second groove 154 may be formed as a partial circumferential notch. The second groove 154 may be positioned such that, with the implantation unit 150 being attached to the casing 110, the second groove 154 is aligned with the first groove 122. The implanting needle attachment includes an implanting needle 152 and a second groove 154. The implanting needle 152 is removably coupled to the casing 110 of the extracting unit. The removeable coupling may be, for example, a friction fit, a detent/groove, a twist-lock, and the like. The second groove 154 is configured to provide access to the lock 130 in a state of the implantation unit 150 being coupled to the casing 110 and overlying the extracting needle 120. The implanting needle 152 may be a splitting needle, formed as a hollow needle with a splitting needle lumen extending therethrough. Further, the splitting needle includes a second distal cutting end that is angled relative to a central axis of the splitting needle. The distal cutting end of the splitting needle is configured to form an opening in a recipient site in order to implant the extracted core. The second distal cutting end has a specific geometry (e.g., the angle of the distal cutting edge) that can prevent tissue from entering the splitting needle while the splitting needle cuts into skin by piercing the skin and gradually pushing the tissue apart, similar to the function of a hypodermic needle.

In the illustrated example, in which the extracting needle 120 is a coring needle and the implanting needle 152 is a splitting needle, the inner diameter of the coring needle and the inner diameter of the splitting needle are substantially the same. That is, the coring needle defines a first diameter that is equal to a second diameter defined by the splitting needle. In other examples, however, the diameters of the coring and splitting needles may be different from one another.

Various components of the hair transplant system 100 may be made of metal, plastic, ceramic, or any other suitable materials. For example, an extracting needle 120 and a pin 140 of the extracting unit and an implanting needle 152 of the implantation unit 150 may be made from medical grade or surgical stainless steel. This allows for easy cleaning of the hair transplant system 100. The extracting needle 120 and implanting needle 152 may be equal or different sizes. In some examples, the hair transplant system 100 may include a plurality of extracting needles 120. In further examples, the hair transplant system 100 may include a plurality of implanting needles 152. Any number of extracting needles 120 and/or implanting needles 152 may be provided, limited only by the size of the donor site, the size of the recipient site, and/or the size of the hair transplant system 100. In some examples, the number of extracting needles 120 is equal to the number of implanting needles. The plurality of extracting needles 120 and/or implanting needles 152 may be arranged in-line or in a circular or other two-dimensional arrangement. A plurality of in-line needles may be arranged in-line or in a circular or other two-dimensional arrangement. A plurality of in-line needles may be spaced so as to extract hairs that are immediately adjacent (e.g., approximately 1 mm apart), evenly spaced, or may be spaced to allow the user to extract hairs that are not immediately adjacent. This can be beneficial because the hairs will be removed at a lower concentration from a greater area, so the patient is not left with a bald spot. The extracting needle 120 and/or implanting needle 152 may also be interchangeable or disposable and replaced after use. In one embodiment the extracting needle 120 and implanting needle 152 are 14 gauge needles. The extracting needle 120 and implanting needle 152 may be exchanged for needles of different sizes.

Now that the general structure of the hair transplant system 100 has been described above, exemplary methods of use will be described below. It should be noted that the methods of use described below are given as examples and are not meant to be limiting in any way.

The hair transplant system 100 can be used to perform multiple different procedures to complete a hair transplant operation on a patient. For example, the hair transplant system 100 is designed to perform an extraction procedure and an implantation procedure. Although any one of these two procedures can be performed individually by the hair transplant system 100, the hair transplant system 100 allows for the two procedures to be done sequentially and repetitively. That is, the hair transplant system 100 can first be used to extract a hair follicle from a donor site of a donor during an extraction procedure. Then, the hair transplant system 100 can be used to implant the hair follicle from the donor site into the recipient site. Finally, once the hair follicle has been implanted into the recipient site, the hair transplant system 100 can be used to repeat this process again and again to complete the hair transplant operation. This process may be repeated, for example, tens, hundreds, or even thousands of times.

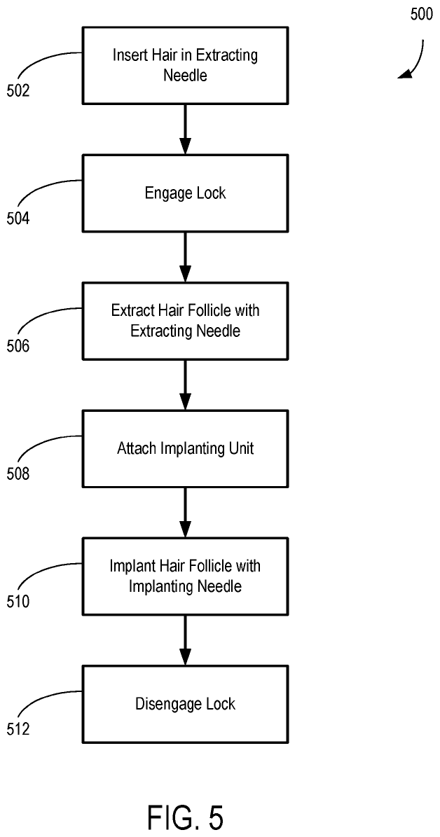

FIG. 5 depicts a method of operation 500 of a hair transplant system. Solely for purposes of explanation, the method 500 will be described as being performed by the hair transplant system 100 of FIGS. 1A-4. The method 500 begins with providing the hair transplant system 100, which may comprise an extracting needle (e.g., 120 of FIG. 1A) having a first groove (e.g., 122 of FIG. 3A) extending longitudinally from a distal end of the extracting needle, the first groove including a notch (e.g., 124 of FIG. 3A). The operation of the hair transplant system 100 is performed with the hair transplant system 100 in an unlocked position. for example as illustrated in FIG. 3A, and with no implantation system attached, for example as illustrated in FIG. 2. Operation 502 includes inserting a hair (e.g., a long hair) through the first groove and out the notch. This may include taking the long hair through the first groove and the end out through the notch, ensuring that the long hair is under tension during harvesting.

With the hair so positioned, operation 504 includes engaging a lock (e.g., 130 of FIG. 1A) disposed on the extracting needle, thereby to close at least a portion of the first groove. This may include sliding the lock from a first position, such as that illustrated in FIG. 3A, to a second position, such as that illustrated in FIG. 3B. With the lock engaged, operation 506 includes extracting a follicle of the hair by piercing a donor site with the extracting needle. A core may be extracted by removing the hair transplant system 100 from the donor site. The hair transplant system 100 may then be prepared for implantation. For example, at operation 506 an implantation unit (e.g., 150 of FIG. 1A) may be attached to the hair transplant system 100, the implantation unit including an implanting needle (e.g., 152 of FIG. 4) and a second groove (e.g., 154 of FIG. 4) configured to align with the first groove in a state of the implantation unit being attached to the hair transplant system 100. With the hair transplant system 100 now prepared for implantation, at operation 510, the lock is disengaged and, at operation 512, implanting the follicle is performed by piercing a recipient site with the implanting needle or by using a sharp scalpel or any relevant sharp object to pierce prior for implanting.

The method 500 may be repeated any number of times. Additionally, as noted above, in some implementations the hair transplant system 100 may include a plurality of extracting needles and implanting needles. In such implementations, the method 500 may proceed as illustrated in FIG. 5. However, in other implementations the hair transplant system 100 may include one extracting needle and a plurality of implanting needles. In such implementations operations 502-506 may be performed a plurality of times equal in number to the plurality of implanting needles, each time extracting a new target follicle. After performing operations 502-506 the plurality of times, operations 508-512 may be performed in order a single time to implant all hair follicles at once. In still other implementations the hair transplant system 100 may include a plurality of extracting needles and one implanting needles. In such implementations operations 502-508 may be performed once, after which operations 510 and 512 may be performed a plurality of times equal in number to the plurality of extracting needles, each time implanting a new follicle.

The method 500 uses one modular needle for harvesting and implantation; thus, follicles can spend very little time outside of the body, resulting in, for example reduced mechanical trauma and the duration that a follicle is outside the body, and thus reduced instances of shedding. Moreover, there may be no need to manipulate the follicle between extraction and implantation. These benefits may further be enhanced by, for example, decreasing metabolic demand of follicles before and after transplantation, and/or hardening hair follicles against cellular stress and apoptosis.

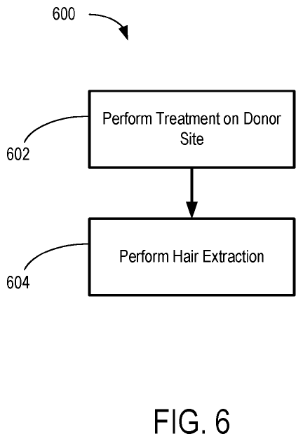

FIG. 6 illustrates an example method of operation 600 to reduce instances of shedding. FIG. 6 includes an operation 602 of performing a treatment on the donor site. The treatment may include cooling of a tissue of the donor site as described in detail below. The treatment may also or alternatively include administering a hair growth inhibitor to the donor site. The treatment may additionally or alternatively include photobiomodulation.

Tissue cooling is a means for decreasing metabolic demand that has been used in other contexts to prevent anagen arrest and hair loss during chemotherapy. The scalp is cooled before, during and for a while after a dose of chemotherapy is given, using a cold cap device at a temperature of 0° C. The scalp temperature is well above 0° C. even when the cold cap is worn. For example, the temperature of hair follicles is likely to be about 10° C. when the cold cap is worn. Human cell metabolic rate measurement is a function of temperature. Metabolism is potently suppressed at temperatures of just 20° C. about 15° C. below the normal scalp temperature. Thus, performing tissue cooling (e.g., as part of operation 602) may decrease the metabolic demand of follicles before and after transplantation.

Eflornithine (sometimes referred to using the trade name Vaniqa) is an FDA-cleared topical hair growth inhibitor that does not kill hair or stop growth, but rather reduces hair growth rate by about 2×. Without wishing to be bound to any one theory of operation, eflornithine may act as a selective inhibitor of ornithine decarboxylase, an enzyme necessary for cell division. By reducing metabolic rate, eflorithine could potentially preserve the anagen phase after hair transplantation. Thus, administering eflornithine or a similar growth inhibitor (e.g., as part of operation 602) may decrease the metabolic demand of follicles before and after transplantation. These results are unexpected, because a lotion used to reduce the appearance of hair would not have been considered to be effective for blocking effluvium (hair loss) after transplantation.

Photobiomodulation (PBM) hardens the active matrix of hair follicles to survive the stress of transplant. PBM uses red and/or near-infrared light to improve mitochondrial functions, staving off cell death and apoptosis, especially in the setting of acute hypoxia. There are at least two distinct molecular pathways for PBM. At 980 nm, TRPV-1 channels are activated to produce a calcium-mediated transient survival response that lasts several hours. This fits with the duration of a hair transplant procedure but is too short to protect transplanted follicles against the stress of hypoxia and inflammation for days. In contrast, mitochondrial respiration is directly enhanced by other wavelengths including ˜670 nm and ˜800 nm. The benefits of 670 and 800 nm PBM persist for about 2 days after a single exposure. PBM takes less than 20 minutes, and could be done by the patient at home during the week after transplantation or could be done in a medical setting.

Cooling, eflornithine and PBM operate by completely different and complementary mechanisms. Cooling reduces metabolic demand, eflornithine blocks mitotic rate, and PBM hardens active cells against stress. Cooling, eflornithine and PBM are therefore synergistic. As such, operation 602 may include any one or a combination of these treatments.

After treatment, at operation 604 hair extraction may be performed. Operation 604 may be the same as or similar to the extraction operations 502-506 illustrated in FIG. 5 and described above, and may be followed by implantation such as by operations 508-512 illustrated in FIG. 5. However, the benefits of operation 602 may be generally applicable and thus operation 604 is not limited to operations described above with regard to FIG. 5. In some implementations, method 600 may be performed for extraction and transplantation of short (e.g., ˜2 mm or shorter) hair.

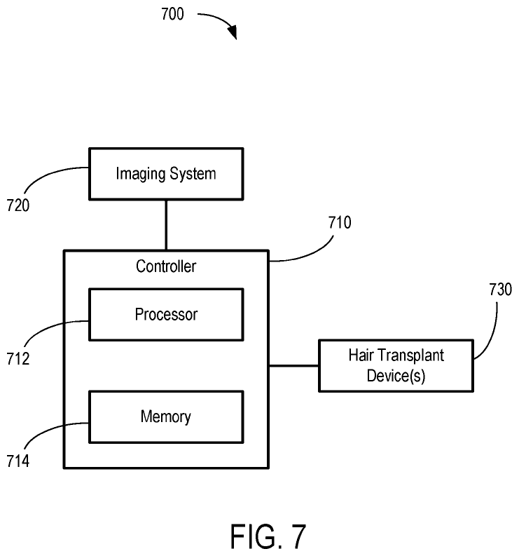

The hair transplant systems and methods described above can be incorporated into an automated hair transplant system. For example, as illustrated in FIG. 7 a hair transplant system 700 may include a controller 710 having one or more inputs, processors, memories, and outputs, and may be configured to operate a single hair transplant device 730 (e.g., the hair transplant system 100 described above) and/or a matrix of hair transplant devices to carry out steps for extracting hair follicles from a donor site and implanting the hair follicles in the recipient site. The system 700 may automate some or all of the operations described above. For example, in some implementations the system 700 may automate extraction and implantation operations, and a user may manually attach the implantation unit to the hair transplant device 730 therebetween. In other operations, the hair transplant device 730 may be configured with devices (e.g., mechanical linkages) to permit the operation of attaching the implantation unit to be automated.

The hair transplant system 700 may include, access, or communicate with one or more user interfaces and/or an imaging system 720, by way of a wired or wireless connection to the inputs. In various implementations, the hair transplant system 700 may include any computing device, apparatus or system configured for carrying out instructions and providing input/output capabilities, and may operate as part of, or in collaboration with other computing devices and sensors/detectors (local and remote). In this regard, the hair transplant system 700 may be a system that is designed to integrate a variety of software and hardware capabilities and functionalities, and/or may be capable of operating autonomously.

The input may include any one or more different input elements, such as a mouse, keyboard, touchpad, touch screen, buttons, and the like, for receiving various selections and operational instructions from a user through touch, movement, speech, etc. The input may also include various drives and receptacles, such as flash-drives. USB drives, CD/DVD drives, and other computer-readable medium receptacles, for receiving various data and information. To this end, input may also include various communication ports and modules, such as Ethernet, Bluetooth, or Wi-Fi, for exchanging data and information with these, and other external computers, systems, devices, machines, mainframes, servers or networks.

In addition to being configured to carry out various steps for operating the hair transplant system, the processor 712 may be configured to execute instructions, stored in the memory 714 in a non-transitory computer-readable media. The instructions executable by the processor 712 may correspond to various instruction for completing a hair transplant procedure (such as those previously described). The memory 714 may be or include a non-volatile medium, e.g., a magnetic media or hard disk, optical storage, or flash memory; a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; on-chip memory; and/or an installation medium where appropriate, such as software media, e.g., a CD-ROM, or floppy disks, on which programs may be stored and/or data communications may be buffered. Although the non-transitory computer-readable media can be included in the memory 714, it may be appreciated that instructions executable by the processor 712 may be additionally or alternatively stored in another data storage location having non-transitory computer-readable media. For example, the hair transplant system 700 may be configured to implement cloud storage.

As used herein, a “processor” may include one or more individual electronic processors, each of which may include one or more processing cores, and/or one or more programmable hardware elements. The processor may be or include any type of electronic processing device, including but not limited to central processing units (CPUs), graphics processing units (GPUs), application-specific integrated circuits (ASICs), field-programmable gate arrays (FPGAs), microcontrollers, digital signal processors (DSPs), or other devices capable of executing software instructions. When a device is referred to as “including a processor,” one or all of the individual electronic processors may be external to the device (e.g., to implement cloud or distributed computing). In implementations where a device has multiple processors and/or multiple processing cores, individual operations described herein may be performed by any one or more of the microprocessors or processing cores, in series or parallel, in any combination.

In some aspects, the processor 712 may be configured to receive and process image data from a subject, such as a donor or a recipient, captured by the imaging system 720 to identify hair follicles and hair follicle orientations within a donor site of the donor and/or to determine implantation locations and necessary implantation angles within a recipient site of the recipient. In some aspects, the processor 712 may access information and data, including video signals, stored in or emitted by the imaging system 720. In some aspects, the imaging system 720 may acquire either a single image or a continuous video signal using, for example, a camera, an infrared scanning system, or any other image capturing or video recording device that can be used to periodically image and/or scan and/or continuously record the subject.

In some instances, the imaging system 720 may be utilized to align the coring needles of the hair transplant devices 730 along a hair shaft or a plurality of hair shafts. In some non-limiting examples, the imaging system 720 can include a camera such as a standard complementary metal-oxide-semiconductor (CMOS) camera, a charge-coupled device (CCD) camera, or an optical coherence tomography (OCT) imaging device. The OCT imaging device may allow for more precise alignment of the coring needles with reference to the hair shafts due to the capability of OCT imaging to see vertically into the tissue. Once the skin cores have been extracted, the hair transplant device 730, under control of the automated hair transplant system 700, may position itself over the recipient site for implantation of the hairs. A computer image may similarly be obtained of the recipient site that may show a natural hair line for the patient and direct where the hairs should be implanted. The ability of the needles to move independently may allow for better shaping and following of a natural hair line. In some instances, the patient may be positioned in a support holder or laying down to limit movement during this process.

The output of the hair transplant system 700 is configured to effectuate the operation of the hair transplant devices 730. As such, the output may include various robotic devices capable of manipulating and operating the hair transplant devices 730 and the interface features thereof, to effectuate extraction of hair follicles from a donor site, creation of openings within the recipient, and implantation of the hair follicles within the openings of the recipient, as described above. As such, a user, such as a doctor or other hair transplant procedure personnel, can interact with a user interface of the hair transplant system 700 to command the automated hair transplant system 700 to effectuate a hair transplant procedure on a subject in accordance with any of the devices and methods described herein.

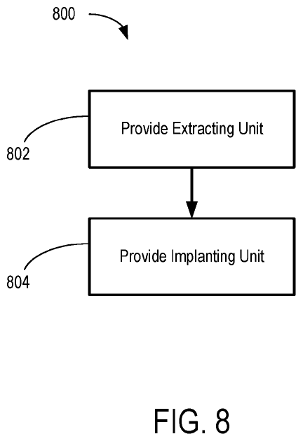

FIG. 8 depicts a method 800 of manufacturing and assembly of a hair transplant system, which may be the hair transplant system 100 illustrated in FIG. 1A. The method 800 includes a first operation 802 of providing an extracting unit (e.g., as shown in FIG. 2). The extracting unit may include a casing and an extracting needle having a first groove extending longitudinally from a distal end of the extracting needle. The first groove includes a notch. A lock is disposed on the extracting needle. The lock is configured to selectively close at least a portion of the first grove. A pin extends through a center of the casing and is configured to cause the extracting needle to selectively take in or eject a hair follicle. The method 800 includes a second operation 804 of providing an implantation unit (e.g., 150 of FIG. 1A). The implantation unit includes an implanting needle and a second groove. The implantation unit is configured to be removably coupled to the casing such that, in a state of the implantation unit being coupled to the casing, the second groove is aligned with the first groove. Operations 802 and 804 may be performed serially in either order, or may be performed in parallel. The hair transplant system may be manufactured in an assembled state, and thus method 500 may be followed by an operation of attaching the implantation unit to the extracting unit.

One or more components of the system may be configured for connection with an automated system, such as, for example a computer-aided manufacturing (CAM) system, for automated use of the hair transplant system 100. The system may additionally be configured for connection with several other similar hair transplant systems, such that an array of hair transplant systems similar to the hair transplant systems 100 is provided to allow for automated extraction and/or implantation of multiple hair follicles in series or simultaneously. In some instances, the system may additionally or alternatively be configured for manual manipulation (e.g., can include a handle).

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. A hair transplant system, comprising:
a casing;
an extracting needle having a first groove extending longitudinally from a distal end of the extracting needle, the first groove including a notch;
a lock disposed on the extracting needle and configured to selectively close at least a portion of the first groove; and
a pin extending through a center of the casing and configured to cause the extracting needle to selectively take in or eject a hair follicle.

2. The system of claim 1, further comprising:
an implanting needle attachment removably coupled to the casing, the implanting needle attachment including an implanting needle and a second groove configured to provide access to the lock in a state of the implanting needle attachment being coupled to the casing and overlying the extracting needle.

3. The system of claim 2, wherein, in a state of the implanting needle attachment being coupled to the casing, the second groove is aligned with the first groove.

4. The system of claim 2, wherein the implanting needle attachment is configured to removably couple to the casing via a friction fit.

5. The system of claim 2, wherein, in a state of the implanting needle attachment being coupled to the casing, actuation of the pin is configured to cause the extracting needle to implant the hair follicle via the implanting needle.

6. The system of claim 1, wherein the lock includes a cylindrical collar configured to receive the extracting needle therein such that the lock is configured to slide along the extracting needle between a first position in which the lock covers the notch and a second position in which the lock does not cover the notch.

7. The system of claim 1, wherein the notch is at least one cm from the distal end of the extracting needle.

8. The system of claim 1, wherein the pin includes a port configured for connection to a vacuum source, and a central lumen in fluid communication with a cavity of the casing configured to receive the hair follicle from a donor site.

9. A hair transplant method, comprising:
providing a hair transplant system comprising an extracting needle having a first groove extending longitudinally from a distal end of the extracting needle, the first groove including a notch;
inserting a hair through the first groove and out the notch;
engaging a lock disposed on the extracting needle, thereby to close at least a portion of the first groove;
extracting a follicle of the hair by piercing a donor site with the extracting needle;
attaching an implantation unit to the hair transplant system, the implantation unit including an implanting needle and a second groove configured to align with the first groove in a state of the implantation unit being attached to the hair transplant system;
piercing a recipient site with the implanting needle; and
disengaging the lock and implanting the follicle.

10. The method of claim 9, wherein the operation of attaching the implantation unit does not include manipulating the follicle.

11. The method of claim 9, further comprising:
prior to the operation of inserting the hair, performing a treatment on the donor site.

12. The method of claim 11, wherein the treatment includes cooling a tissue of the donor site.

13. The method of claim 11, wherein the treatment includes administering a hair growth inhibitor to the donor site.

14. The method of claim 11, wherein the treatment includes photobiomodulation.

15. The method of claim 9, wherein the operation of engaging the lock includes tensioning the hair.

16. The method of claim 9, wherein the hair transplant system further comprises a casing and a pin extending through a center of the casing, and wherein the operation of extracting the follicle includes applying a vacuum via the pin.

17. The method of claim 9, wherein the hair transplant system further comprises a pin extending through a center of the casing, and wherein the operation of implanting the follicle includes actuating the pin.

18. The method of claim 9, wherein the operation of engaging the lock includes sliding the lock along the extracting needle from a first position in which the notch is exposed to a second position in which the hair passes through the notch and the lock is advanced to close the groove in the extracting needle.

19. The method of claim 9, further comprising:
after the operation of disengaging the lock and implanting the follicle, removing the hair transplant system from the recipient site.

20. A method of manufacturing a hair transplant system, comprising:
providing an extracting unit, the extracting unit including;
a casing,
an extracting needle having a first groove extending longitudinally from a distal end of the extracting needle, the first groove including a notch,
a lock disposed on the extracting needle and configured to selectively close at least a portion of the first groove, and
a pin extending through a center of the casing and configured to cause the extracting needle to selectively take in or eject a hair follicle; and
providing an implantation unit, the implantation unit including an implanting needle and a second groove.
wherein the implantation unit is configured to be removably coupled to the casing such that, in a state of the implantation unit being coupled to the casing, the second groove is aligned with the first groove.
